(19) United States
(12) Patent Application Publication
(10) Pub. No.: US 2007/0231317 A1
(43) Pub. Date: Oct. 4, 2007

(54) S100 Protein Inhibitors for Treating Leukemia

(75) Inventors: Philippe Tessier, Quebec (CA); Karen Vandal, Quebec (CA); Pascal Rouleau, Quebec (CA); Carle Ryckman, Quebec (CA)

Correspondence Address: BIRCH STEWART KOLASCH & BIRCH, PO BOX 747, FALLS CHURCH, VA, 22040-0747, US

(73) Assignee: UNIVERSITE LAVAL, QUEBEC (CA)

(21) Appl. No.: 11/632,166

(22) PCT Filed: Jul. 13, 2005
(86) PCT No.: PCT/CA05/01089

§ 371 Date: Mar. 12, 2007

Related U.S. Application Data
(60) Provisional application No. 60/587,033, filed on Jul. 13, 2004.

(51) Int. Cl. A61K 39/395; A61K 31/7105; A61K 38/00

(52) U.S. Cl. 424/130.1; 514/2; 514/44

(57) ABSTRACT

The present invention is directed to compounds, composition and method for reducing or inhibiting the differentiation or development of progenitor blood cells into leukemia cells, Particularly, the invention describes the use of inhibitors of S100 proteins, including myeloid related proteins (MRP). Inhibition of the activity or synthesis of S100 proteins results in the reduction or inhibition of the production or proliferation of leukemia cells.

### BACKGROUND OF THE INVENTION

a) Field of the Invention

The present invention relates to compositions and treatments for patients suffering of leukemia. Particularly, the invention relates to the inhibition of proliferation of hematopoietic cells leading to pathologic blood cell proliferation conditions, namely leukemia.

b) Description of the Prior Art

Leukemia is a malignant cancer of the bone marrow and blood. It is characterized by the uncontrolled growth of blood cells. The common types of leukemia are divided into four categories: acute or chronic myelogenous, involving the myeloid elements of the bone marrow (white cells, red cells, megakaryocytes) and acute or chronic lymphocytic, involving the cells of the lymphoid lineage.

Acute leukemia is a rapidly progressing disease that results in the massive accumulation of immature, functionless cells (blasts) in the marrow and blood. The marrow often can no longer produce enough normal red and white blood cells and platelets. Anemia, a deficiency of red cells, develops in virtually all leukemia patients. The lack of normal white cells impairs the body's ability to fight infections. A shortage of platelets results in bruising and easy bleeding. In contrast, chronic leukemia progresses more slowly and leads to unregulated proliferation and hence marked overexpansion of a spectrum of mature (differentiated) cells. In general, acute leukemia, unlike the chronic form, is potentially curable by elimination of the neoplastic clone. Chronic myeloid leukemia (hereinafter CML) is one of the four major types of leukemia encountered by humans, the others being acute lymphocytic leukemia, acute myeloid leukemia (AML) and chronic lymphocytic leukemia. Acute leukemias are characterised by a rapidly progressive, fatal course if untreated; acute myeloid leukemia (AML) strikes predominantly adults. Chronic leukemias are indolent, often asymptomatic diseases in which median survivals measures in years. Cancerous cells crowd out the normal cells in the bone marrow and lymph nodes. Anemia develops in the patient and the number of normal white cells and platelets in the patient's blood decreases, whereas the total white cell count increases due to the proliferation of abnormal white cells. The level and activity of antibodies also decrease. As a result, the patient's immune system becomes compromised. It is more common for leukemia sufferers to die from consequences of the compromised immune system, e.g. infections, than from the leukemia itself.

Chronic myeloid leukemia (CML) accounts for approximately 20% of all leukemia. The majority of the patients with CML have evidences of the Philadelphia chromosome, a (9:22) chromosomal translocation. This translocation link the break cluster region (Bcr) to the c-abl tyrosine kinase, resulting in bcr-abl fusion gene and protein. The Philadelphia chromosome is also present in 5% of adult AML.

Although bcr-abl confers immortality to the cell, it is not sufficient for growth factor-independent growth of the tumor cell. Acquisition of several molecular abnormalities is required for these cells to become completely independent of growth factors. The development of growth autonomy could possibly result from the inappropriate expression and secretion of growth factors by the tumor cells, leading to the establishment of an autocrine loop, as found in other hematopoietic malignancies such as multiple myeloma. In fact, expression of bcr-abl in several different myeloid cell lines has been associated with the induction of IL-3/GM-CSF autocrine loops. Other growth factors are also suspected to induce autocrine loops. Knowledge of the growth factors inducing proliferation of these cells is therefore important since it could suggest new therapeutic avenues to cure leukemia.

Treatment of leukemia is very complex and depends upon the type of leukemia. Tremendous clinical variability among remissions is also observed in leukemic patients, even those that occur after one course of therapy. Patients who are resistant to therapy have very short survival times, regardless of when the resistance occurs.

Standard treatment for leukemia usually involves chemotherapy and/or bone marrow transplantation and/or radiation therapy. The two major types of bone marrow transplants are autologous (uses the patient's own marrow) and allogeneic (uses marrow from a compatible donor). Radiation therapy, which involves the use of high-energy rays, and chemotherapy are usually given before bone marrow transplantation to kill all leukemic cells. In the cure for CML, bone marrow transplantation can be clearly curative. However, only 30% to 40% of patients with CML have an appropriate donor. Beyond that, the mortality from the procedure ranges from 20% to 30%, depending on the age of the recipient. Finally, this procedure is quite expensive.

Chemotherapy in leukemia may involve a combination of two or more anti-cancer drugs. Approximately 40 different drugs are now being used in the treatment of leukemia, either alone or in combination. Some common combinations include cytarabine with either doxorubicin or daunorubicin or mitoxantrone or thioguanine, mercaptopurine with methotrexate, mitoxantrone with etoposide, asparaginase with vincristine, daunorubicin and prednisone, cyclophosphamide with vincristine, cytarabine and prednisone, cyclophosphamide with vincristine and prednisone, daunorubicin with cytarabine and thioguanine and daunorubicin with vincristine and prednisone.

Other treatments for leukemia also include the reversal of multidrug resistance, involving the use of agents which decrease the mechanisms allowing the malignant cells to escape the damaging effects of the chemotherapeutic agent (and leads to refractoriness or relapses); and biological therapy, involving the use of substances known as biological response modifiers (BRMs). These substances are normally produced in small amounts as part of the body's natural response to cancer or other diseases. Types of BRMs include monoclonal antibodies, in which toxins are attached to antibodies that react with the complementary antigen carried by the malignant cells; and cytokines (e.g. interferons, interleukins, colony-stimulating factors CSFs) which are naturally occurring chemicals that stimulate blood cell production and help restore blood cell counts more rapidly after treatment. Examples of these drugs include multidrug resistance reversing agent PSC 833, the monoclonal antibody Rituxan™ and the following cytokines: erythropoietin and epoetin, which stimulate the production of red cells; G-CSF, GM-CSF, filgrastim, and sargramostim which stimulate the production of white cells; and thrombopoietin, which stimulate the production of platelets.

Many nucleoside analogues have been found to possess anticancer activity. Cytarabine™, Fludarabine™, Gemcitabine™ and Cladribine™ are some examples of nucleoside analogues which are currently important drugs in the treatment of leukemia. βL-OddC ((−)-β-L-Dioxolane-Cytidine, Troxatyl™, from Shire BioChem Inc.) is also a nucleoside analogue which was first described as an antiviral agent by Belleau et al. (EP 337713) and was shown to have potent antitumor activity (K. L. Grove et al., Cancer Res., 55(14), 3008-11, 1995; K. L. Grove et al., Cancer Res., 56(18), 4187-4191, 1996, K. L. Grove et al., Nucleosides Nucleotides, 16:1229-33, 1997; S. A Kadhim et al., Can. Cancer Res., 57(21), 4803-10, 1997). In clinical studies, β-L-OddC has been reported to have significant activity in patients with advanced leukemia (Giles et al., J. Clin. Oncology, Vol 19, No 3, 2001).

More recently, STI-571 (Gleevec™, imatinib mesylate, from Novartis Pharmaceuticals Corp.), a Bcr-Abl tyrosine kinase inhibitor has shown significant antileukemic activity and specifically in chronic myelogenous leukemia. STI-571 has become a promising therapy in the group of patients targeting Bcr-Abl tyrosine kinase inhibition. However, despite significant hematologic and cytogenic responses, resistance occurs particularly in the advanced phases of chronic myelogenous leukemia.

Therefore, there is a great need for the further development of agents for the treatment of blood related cancers.

From the state of the art described herein above, there is still a great need for agents and compounds allowing the treatment and prevention of leukemia or of differentiation and development disorders of blood cells and blood progenitor cells.

### SUMMARY OF THE INVENTION

One aim of the present invention is to provide a composition for modulating the differentiation, the growth or the development of blood cells or progenitor blood cells, such as for example bone marrow stem cells, in a human or an animal comprising an effective amount of at least one inhibitor of a compound of S100 protein family with a physiologically acceptable carrier. It is understood here that the modulation can be as well reducing or inhibiting the differentiation or development of said progenitor blood cells into physiologically pathological cells, as for example, but not limited to, leukemia cells.

The progenitor stem cells, in accordance with another object of the present invention, can be inhibited to differentiate and/or proliferate into leukocytes, neutrophils, eosinophils, basophils, lymphocytes, macrophages, or monocytes.

In one particular object, the composition of the present invention can comprise at least an antibody or a fragment thereof, a peptide, an anti-mRNA, an RNAi, siRNA, an inhibitor of transcription or translation of S100 protein, or a binding inhibitor binding a targeted S100 protein or its natural binding site for inhibiting the differentiation, development or proliferation of a blood stem cell into a physiologically pathological cell.

The S100 protein can be selected, but not limited to, from the group consisting of MRP, S100A8, S100A9, S100A12, or a derivative, a fragment or an analog thereof, or dimers thereof. The dimers can be found alternatively, or depending of the needs, under forms of homodimers or heterodimers.

Another aim of the present invention is to provide also a method for modulating the physiology of pathological blood cells in a human or an animal comprising the step of administering to a human or animal in need thereof a composition comprising a pharmaceutically acceptable carrier and an effective amount of at least one inhibitor of a compound of the S100 protein family.

In accordance with the present invention there is provided the use of an inhibitor of a compound or member of the S100 protein family in the preparation of a compound or a composition for modulating differentiation or development of progenitor blood cells in a human or an animal.

Accordingly, it is an object of the present invention to provide novel procedures and compositions for alleviation of leukemia in mammalian patients.

It is a further object of the invention to provide procedures and compositions which, on suitable administration to a leukemia suffering patient, will significantly postpone the need for subjecting the patient to chemotherapy. It has now been discovered that the proliferation of leukemia cells from patients suffering from leukemia, such as for example, but not limited to, acute and chronic myeloid leukemia (AML and CML), is reduced by the administration of certain inhibitors of S100 proteins, such as antibodies against MRP, S100A8, S100A9 and S100A12, alone or together, or related S100 compounds. It will be recognized to those skilled in the art that the object of the present invention can be performed through different ways, including, but not exclusively, by in vivo, in vitro, ex-vivo and in situ ways.

Thus, one aspect of the present invention relates to the discovery that inhibitors of S100 proteins treat and/or reduce the level of leukemia cells in patients.

The present invention also relates to the use of inhibitors of S100 proteins, such as Myeloid Related Proteins (MRP), and/or functional derivatives thereof, in the manufacture of a pharmaceutical composition for the treatment of leukemia, AML or CML.

In yet another aspect, the invention relates to pharmaceutical compositions for the treatment of leukemia, AML or CML, comprising inhibitors of S100 proteins, related MRP compounds and/or derivatives thereof, as active ingredients, optionally together with pharmaceutically acceptable carrier and/or excipient and/or adjuvant.

In yet other aspects of the invention, a method is provided for the treatment of leukemia, AML or CML by administering to a patient in need thereof, a leukocyte reducing amount of at least one inhibitor of S100 proteins or MRP related protein, either alone or together with a pharmaceutically acceptable carrier.

The invention provides a method and a pharmaceutical composition for treating leukemia, inhibiting the growth or inhibiting the proliferation of leukemia cells with little or no undesired side effects on normal cells, and extending life expectancy of a animal having leukemia. Accordingly, one aspect of the invention provides a method of treating an animal suffering from leukemia comprising the step of administering to the animal in need thereof a safe and effective amount of at least one inhibitor of S100 protein or MRP or derivative thereof.

Another aspect of the invention provides a method of inhibiting the proliferation of leukemia cells comprising the step of treating said cells with an effective amount of an inhibitor of an S100 protein or a derivative thereof, as defined herein. Optionally, one or more potentiators and chemotherapeutic agents can be used in combination with an inhibitor of S100 protein.

Yet another aspect of the invention provides a method of inhibiting the growth of leukemia cells comprising the step of treating said cells with an effective amount of inhibitor of an S100 protein or derivative thereof, as defined herein. Optionally, one or more potentiators and chemotherapeutic agents are used in combination with a S100 protein inhibitor to inhibit the growth of leukemia cells.

Still another aspect of the invention provides a method of extending the life expectancy of a animal having leukemia comprising the step of administering to the animal an effective amount of S100 protein inhibitors, as defined above, whereby the life expectancy of the animal is extended beyond the expected life expectancy of a comparable animal having a comparable degree of leukemia development not being treated with a S100 protein inhibitor. Optionally, one or more potentiators and chemotherapeutic agents are used in combination with the inhibitor of the S100 protein to extend the life expectancy of the animal.

For the purpose of the present invention the following terms are defined below.

The expression “pharmaceutically acceptable” in relation to a compound, a carrier or an excipient, is intended to mean that such compound, carrier or excipient is suitable for use with humans and/or animals without undue adverse side effects (such as toxicity, irritation, and allergic response) commensurate with a reasonable benefit/risk ratio.

As used herein, the term “animal” includes any warm blooded animal and preferably mammals, such as, but not limited to, human.

As used herein, the expression “safe” and/or “effective amount” or “therapeutically effective amounts” refers to the quantity of a component which is sufficient to yield a desired therapeutic response without undue adverse side effects (such as toxicity, irritation, or allergic response) commensurate with a reasonable benefit/risk ratio when used in the manner of this invention. The specific “safe and effective amount” will vary with such factors as the particular condition being treated, the physical condition of the patient, the type of animal being treated, the duration of the treatment, the nature of concurrent therapy (if any), and the specific formulations employed and the structure of the compounds or its derivatives.

As used herein, a “pharmaceutical carrier” is a pharmaceutically acceptable solvent, suspending agent or vehicle for delivering a compound of the S100 protein to an animal or human. The carrier may be liquid or solid and is selected according to the mode of administration in mind.

As used herein, “cancer” or “leukemia” refers to neoplastic diseases which attack normal healthy blood cells, or bone marrow which produces blood cells, which are found in animals. Types of leukemia targeted through the application of the present invention can be, but not limited to, acute or chronic leukemia, eosinophilic, lymphoblastic, lymphocytic, myeloblastic, myelocytic, myelofibrotic, myelogenous, myelomonocytic, pregnancy related, megakaryotic, or promyelocytic leukemia.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease, i.e., acute or chronic; (2) the type of cell involved; myeloid (myelogenous), lymphoid (lymphogenous), or monocytic; and (3) the increase or non-increase in the number for abnormal cells in the blood-leukemic or aleukemic (subleukemic).

As used herein, the term “susceptible to treatment” refers to a leukemia which can be treated with an inhibitor of the S100 protein family according to the methods of the invention. For example, leukemia which is susceptible to treatment will respond favorably to chemotherapy with at least one inhibitor of the S100 protein. A favorable response would include prolongation of the life expectancy of a animal having the leukemia, inhibition of the proliferation of leukemia cells, inhibition of a growth of leukemia cells, reduction in the rate of disease progression in the animal, remission or regression of the disease in the animal, and/or improvement in the quality of life of a animal having leukemia. Treatment with S100 protein inhibitors can be applied to persons exposed to ionizing radiations and certain chemicals, as for example benzene, some antineoplastic drugs. Patients with some genetic defects, such as Down syndrome, Fanconi's anemia, which are predisposed to leukemia, can be subjected to the compounds, composition and treatment as described herein.

As used herein, the term “inhibitor” is intended to mean any product, compound or agent that can physiologically act in inhibiting or reducing the production or the activity of an S100 protein or a derivative thereof. The inhibitor can be under form of a peptide, a polypeptide, a protein, such as, but not limited to, an antibody of a binding fragment thereof, a DNA fragment, an RNA, an siRNA, an iRNA, or any other natural or synthetic compound. This can be an organic or inorganic compound, product or agent.

### BRIEF DESCRIPTION OF THE DRAWINGS

FIG. 1 illustrates the accumulation of leucocytes induced by injection of S100 proteins;

FIG. 2 illustrates induction of neutrophilia by injection of human S100A12 in mouse;

FIG. 3 illustrates induction of proliferation of bone marrow cells after administration of S100A8, S100A9 and S100A12 in mouse

FIG. 4 illustrates proliferation of human leukemia cells stimulated by S100A8, S100A9 and S100 A12 proteins; and

FIG. 5 illustrates inhibition of proliferation of human leukemia cells using antibodies against S100A8 and S100A9.

### DESCRIPTION OF THE PREFERRED EMBODIMENT

The present invention, with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided a compound, composition and method for treating and/or reducing uncontrolled or pathological differentiation or development of blood cells. Particularly, the compound of the present invention relates to inhibitors of the S100 protein family, including, but not limited to, inhibitors of MRPs, S100A8, S100A9, derivatives or analogs thereof. The S100 proteins can be targeted for inhibition by the inhibitors under different forms including homodimers or heterodimers.

The S100 proteins family comprises 19 members of small (10 to 14 kDa) acidic calcium-binding proteins. They are characterized by the presence of two EF-hand type calcium-binding motifs, one having two amino acids more than the other. These intracellular proteins are involved in the regulation of protein phosphorylation, enzymatic activities, Ca²⁺ homeostasis, and intermediate filaments polymerisation. S100 proteins generally exist as homodimers, but some can form heterodimers. More than half of the S100 proteins are also found in the extracellular space where they exert cytokine-like activities through specific receptors; one being recently characterized as the receptor for advanced glycosylation end-products (RAGE). S100A8, S100A9, and S100A12 belong to a subset of the S100 protein family called Myeloid Related Proteins (MRPs) because their expression is almost completely restricted to neutrophils and monocytes, products of the myeloid precursors.

High concentrations of MRP in serum occurs in pathologies associated with increased numbers of circulating neutrophils or their activity. They are also expressed at very high levels in the synovial fluid and plasma of patients suffering from rheumatoid arthritis and gout. High levels of MRPs (up to 13 μg/ml) are also known as being present in the plasma of chronic myeloid leukemia and chronic lymphoid leukemia patients. The presence of these proteins even preceded the apparition of leukemia cells in the blood of relapsing patients. The extracellular presence of S100A8/A9 suggests that the MRPs can be released either actively or during cell necrosis. Like IL-1 and FGFβ, MRPs are expressed in the cytosol, implying they are secreted via an alternative pathway.

Hematopoiesis refers to the proliferation and differentiation of blood cells. The major site of hematopoiesis in humans, after about 20 weeks of fetal life, is the bone marrow. Blood cells develop from multipotent stem cells that are usually located in the bone marrow. These stem cells have the capacity to proliferate and differentiate. Proliferation maintains the stem cell population, whereas differentiation results in the formation of various types of mature blood cells that are grouped into one of three major blood cell lineages, the lymphoid, myeloid or erythroid cell lineages. The lymphoid lineage is comprised of B cells and T cells, which collectively function in antibody production and antigen detection, thereby functioning as a cellular and humoral immune system. The myeloid lineage, which is comprised of monocytes (macrophages), granulocytes (including neutrophils), and megakaryocytes, monitors the bloodstream for antigens, scavenges antigens from the bloodstream, fights off infectious agents, and produces platelets, which are involved in blood clotting. The erythroid lineage is comprised of red blood cells, which carry oxygen throughout the body.

The stem cell population constitutes only a small percentage of the total cell population in the bone marrow. The stem cells as well as committed progenitor cells destined to become neutrophils, erythrocytes, platelets, etc., may be distinguished from most other cells by the presence of the particular progenitor “marker” antigen that is present on the surface of these stem/progenitor cells.

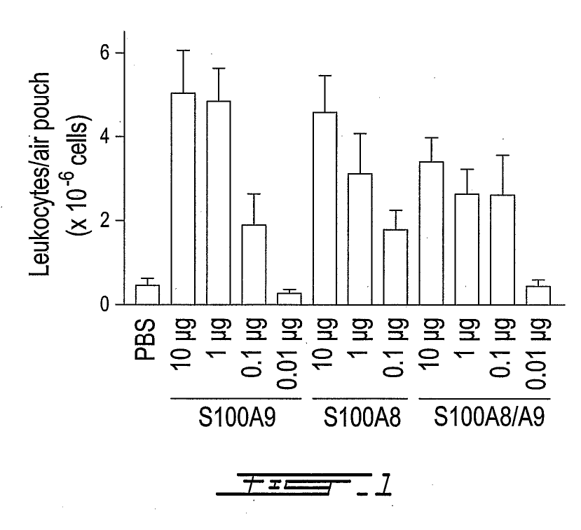

Neutrophils differentiate from stem cells through a series of intermediate precursor cells, which can be distinguished by their microscopic morphological appearance, including such characteristics as the size of their nuclei, the shape of their nuclei, cell size, nuclear/cytoplasmic ratio, presence/absence of granules, and staining characteristics (see FIG. 1). Initially, the multipotent stem cell, which cannot be measured directly in vitro, gives rise to myeloid “progenitor cells” that generate precursors for all myeloid cell lines. The first myeloid progenitor is designated CFU-GEMM for “colony forming unit—granulocyte, erythroid, macrophage and megakaryocyte”. The CFU-GEMM progenitor, in turn, will give rise to a CFU-GM progenitor cell, which is otherwise known as “colony forming unit—granulocyte and macrophage”. In all of these descriptive terms, “colony” refers to a cell that is capable of giving rise to more than 50 cells as measured in 14 day in vitro assays for clonal growth, under conditions as set forth in Example 5 described hereinbelow. These cells will divide at least six times.

The CFU-GM is a committed progenitor—in other words, it is committed to differentiating into granulocytes and macrophages only. It is neither capable of differentiating into other types of cells nor is it capable of dedifferentiating into earlier stage progenitor cells. The CFU-GM progenitor cell may then differentiate into a myeloblast. The time required for differentiation from a CFU-GEMM to a myeloblast is believed to be about 1-4 days. A myeloblast is the first of the series of cells that may be referred to as “precursors” to the neutrophils, as such cells, once allowed to fully develop (differentiate), can only form neutrophils, which are only capable of undergoing fewer than six cell divisions and, therefore, do form colonies in in vitro colony assays as described previously.

Once differentiation has progressed to the myeloblast stage, the myeloblasts undergo terminal differentiation into promyelocytes, which, in turn, differentiate into myelocytes over a course of about 4-6 days. Within another 5 days or so, myelocytes differentiate into metamyelocytes, which, in turn, differentiate into banded neutrophils. These banded neutrophils finally differentiate into mature, segmented neutrophils, which have a half-life of about 0.3 to 2 days. The term “progenitor” will be used to refer to stem cells and cells which can form colonies. “Precursor” will be used to refer to myeloblasts, promyelocytes and myelocytes and, in some instances, metamyelocytes and banded neutrophils, also.

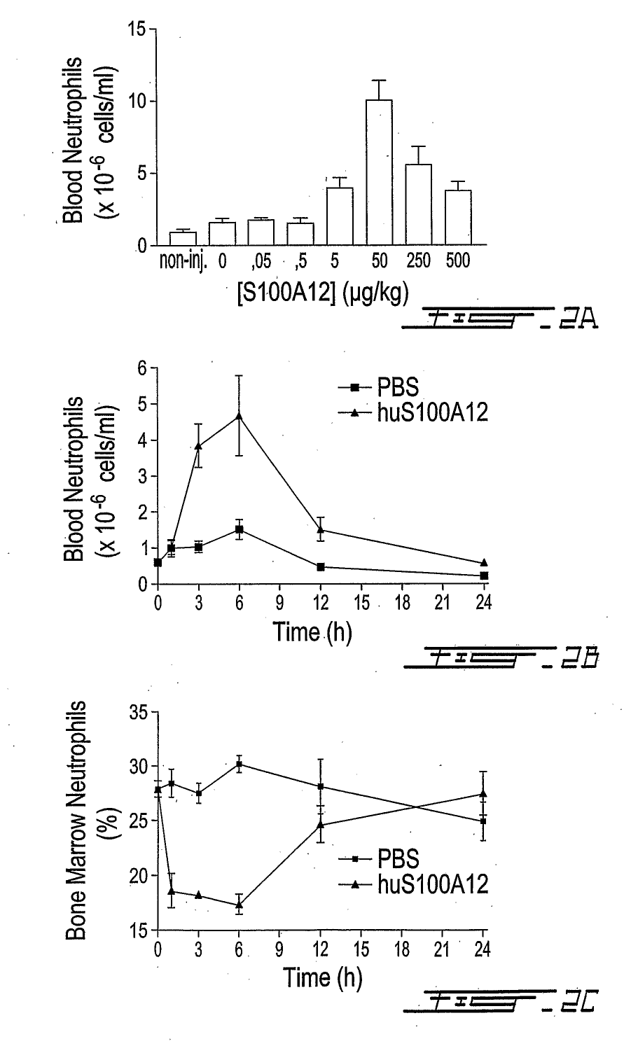

S100A8, S100A9, and S100A12 induce an inflammatory reaction when injected in the murine air pouch model. In this model, sterile air is injected subcutaneously under the dorsum of mice on days 0 and 3. On day 7, an enclosed environment is formed in which it is possible to inject pro-inflammatory products. Injection of S100A8, S100A9, S100A12, or S100A8/A9 in the air pouch leads to the accumulation within 3 hrs of up to 8×10⁶ leukocytes (FIG. 1). Leukocytes recruited consists of neutrophils (80%) and monocytes. The total number of neutrophils circulating in the peripheral blood of a mouse is estimated at 2.5×10⁶ cells. Injection of S100 proteins in the air pouch leads to the migration of more neutrophils than were contained in the blood, suggesting that it induces the release of neutrophils from the bone marrow. This is confirmed by i.v. injections of S100 proteins which leads to the release of neutrophils from the bone marrow to the peripheral blood (FIG. 2). These results demonstrate that S100 proteins are pro-inflammatory and induce the release of neutrophils from the bone marrow. More importantly, the concentrations at which they exerted these functions are similar to those found in the plasma of patients suffering, for example but not limited to, from AML and CML.

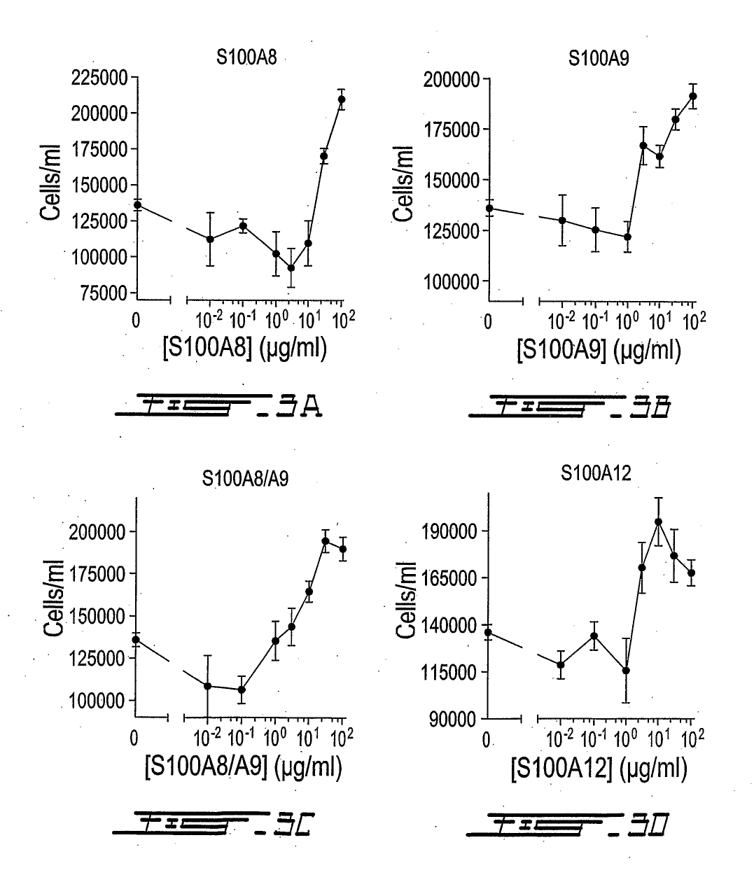

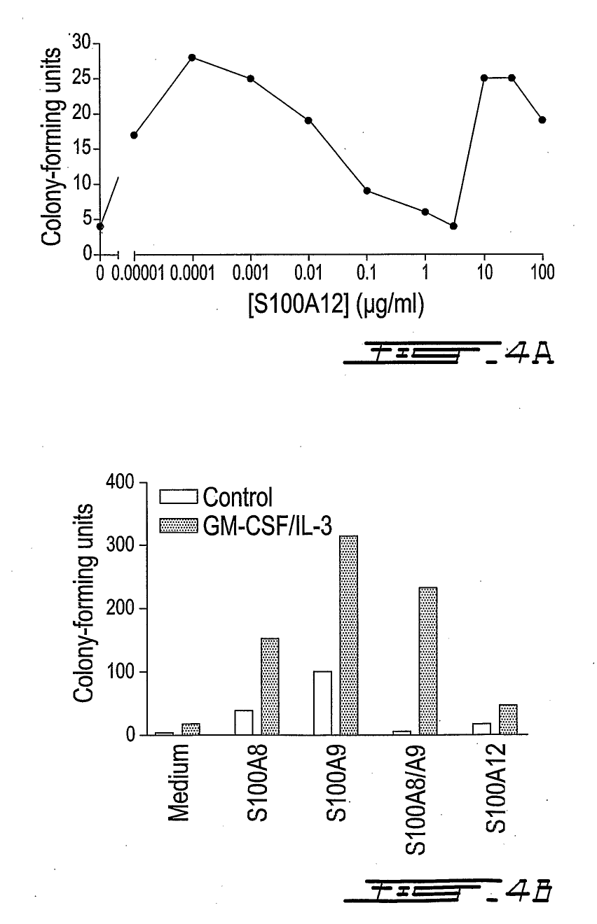

Incubation of bone marrow cells with S100A8, S100A9, S100A8/A9, or S100A12 results in an increase of proliferation (FIG. 3). This proliferation also occurs when bone marrow cells from AML and CML patients are incubated with S100A8, S100A9, and S100A12 proteins (FIG. 4). It is considered that this proliferation occurs at concentrations similar to those detected in the serum of the same patients. It is also considered that S100 proteins can be secreted, depending on the physiological circumstances, by leukemia cells itself to stimulate their proliferation in an autocrine fashion.

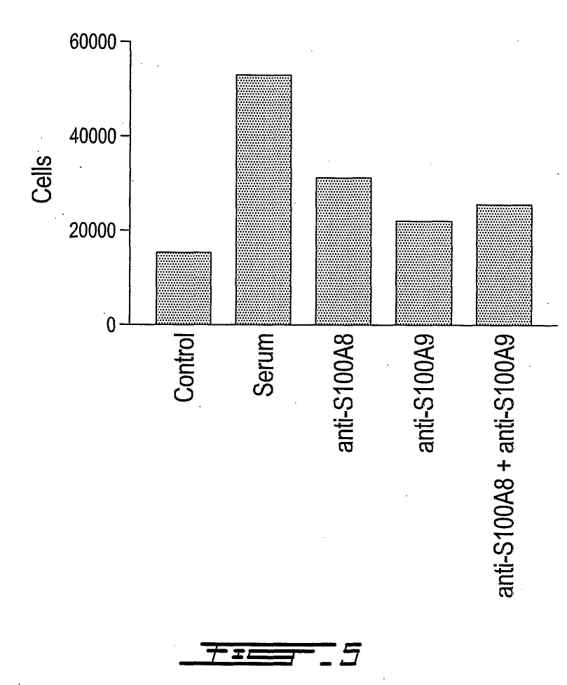

It has been pointed up through the present invention that inhibition of S100 proteins directly or indirectly, through their activity, binding, expression or secretion, significantly reduce the number of progenitor cells ongoing differentiation or development into aberrant cancer blood cells. Indeed, inhibition of S100A8 and S100A9 activity in the serum of AML patients blocks the proliferation of their leukemia cells (FIG. 5).

The activity, differentiation or development of a S100 protein can be inhibited or reduced by using, for example, an antibody, preferably a monoclonal antibody capable of binding the S100 protein without affecting other target in the treated organism. Other approaches, such as peptide inhibitors, drugs, anti-mRNA, siRNA, RNAi, transcription or translation inhibitors, can be used as well to perform the method of the present invention which consists in inhibiting or blocking the production or the activity of S100 proteins, and therefore the differentiation or development of progenitor blood cells into leukocytes having cancer behavior.

In some embodiments of the invention, S100 protein inhibitors can be used in combination with one or more other anti-inflammatory, anti-viral, anti-fungal, amoebicidal, trichomonocidal, analgesic, anti-neoplastic, anti-hypertensive, anti-microbial and/or steroid drugs to treat leukemia.

Other potentiators which can be used with an inhibitor of S100 protein, and optionally a chemotherapeutic agent, in the methods of the invention include macrophage colony-stimulating factor (M-CSF), 7-thia-8-oxoguanosine, 6-mercaptopurine, vitamin A (retinol), monensin, an anti-sense inhibitor of the RAD51 gene, bromodeoxyuridine, dipyridamole, indomethacin, a monoclonal antibody, an anti-transferrin receptor immunotoxin, metoclopramide, N-solanesyl-N,N′-bis(3,4-dimethoxybenzyl)ethylenediamine, leucovorin, heparin, N-[4-[(4-fluorphenyl)sulfonly]phenyl]acetamide, heparin sulfate, cimetidine, a radiosensitizer, a chemosensitizer, a hypoxic cell cytotoxic agent, muramyl dipeptide, vitamin A, 2′-deoxycoformycin, a bis-diketopiperazine derivative, and dimethyl sulfoxide.

The chemotherapeutic agents which can be used with an inhibitor of S100 protein and an optional potentiator are generally grouped as DNA-interactive agents, antimetabolites, tubulin-interactive agents, hormonal agents and others such as asparaginase or hydroxyarea. Each of the groups of chemotherapeutic agents can be further divided by type of activity or compound. For a detailed discussion of chemotherapeutic agents and their method of administration, see Dorr, et al, Cancer Chemotherapy Handbook, 2d edition, pages 15-34, Appleton & Lange (Connecticut, 1994) the disclosure of which is hereby incorporated by reference.

DNA-interactive agents include the alkylating agents, e.g. cisplatin, cyclophosphamide, altretamine; the DNA strand-breaking agents, such as bleomycin; the intercalating topoisomerase II inhibitors, e.g., dactinomycin and doxorubicin; the nonintercalating topoisomerase II inhibitors, such as etoposide and teniposide; and the DNA minor groove binder plicamycin.

The identity of the chemotherapeutic agent, the pharmaceutical carrier and the amount of compound administered will vary widely depending on the species and body weight of mammal and the type of leukemia being treated. The dosage administered will vary depending upon known factors, such as the pharmaco-dynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment and the desired therapeutic effect.

One can use topical administration to deliver a compound of the invention by percutaneous passage of the drug into the systemic circulation of the patient. The skin sites include anatomic regions for transdermally administering the drug, such as the forearm, abdomen, chest, back, buttock, and mastoidal area. The compound is administered to the skin by placing on the skin either a topical formulation comprising the compound or a transdermal drug delivery device that administers the S100 protein inhibitor. In either embodiment, the delivery vehicle is designed, shaped, sized, and adapted for easy placement and comfortable retention on the skin.

A variety of transdermal drug delivery devices can be employed with the compounds of this invention. For example, a simple adhesive patch comprising a backing material and an acrylate adhesive can be prepared. The drug and any penetration enhancer can be formulated into the adhesive casting solution. The adhesive casting solution can be cast directly onto the backing material or can be applied to the skin to form an adherent coating. See, e.g., U.S. Pat. Nos. 4,310,509; 4,560,555; and 4,542,012.

In other embodiments, the compound of the invention will be delivered using a liquid reservoir system drug delivery device. These systems typically comprise a backing material, a membrane, an acrylate based adhesive, and a release liner. The membrane is sealed to the backing to form a reservoir. The drug or compound and any vehicles, enhancers, stabilizers, gelling agents, and the like are then incorporated into the reservoir. See, e.g., U.S. Pat. Nos. 4,597,961; 4,485,097; 4,608,249; 4,505,891; 3,843,480; 3,948,254; 3,948,262; 3,053,255; and 3,993,073.

Matrix patches comprising a backing, a drug/penetration enhancer matrix, a membrane, and an adhesive can also be employed to deliver a compound of the invention transdermally. The matrix material typically will comprise a polyurethane foam. The drug, any enhancers, vehicles, stabilizers, and the like are combined with the foam precursors. The foam is allowed to cure to produce a tacky, elastomeric matrix which can be directly affixed to the backing material. See, e.g., U.S. Pat. Nos. 4,542,013; 4,460,562; 4,466,953; 4,482,534; and 4,533,540.

Also included within the invention are preparations for topical application to the skin comprising a S100 protein inhibitor of the invention, together with a non-toxic, pharmaceutically acceptable topical carrier. These topical preparations can be prepared by combining an active ingredient according to this invention with conventional pharmaceutical diluents and carriers commonly used in topical dry, liquid, and cream formulations. Ointment and creams may, for example, be formulated with an aqueous or oily base with the addition of suitable thickening and/or gelling agents. Such bases may include water and/or an oil, such as liquid paraffin or a vegetable oil, such as peanut oil or castor oil. Thickening agents that may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and will, in general, also include one or more of the following: stabilizing agents, emulsifying agents, dispersing agents, suspending agents, thickening agents, coloring agents, perfumes, and the like. Powders may be formed with the aid of any suitable powder base, e.g., talc, lactose, starch, and the like. Drops may be formulated with an aqueous base or non-aqueous base also comprising one or more dispersing agents, suspending agents, solubilizing agents, and the like. Topical administration of inhibitors of the invention may also be preferred for treating diseases such as skin cancer and fungal infections of the skin (pathogenic fungi typically express telomerase activity).

The S100 inhibitors of the present invention can also be delivered through mucosal membranes. Transmucosal (i.e., sublingual, buccal, and vaginal) drug delivery provides for an efficient entry of active substances to systemic circulation and reduces immediate metabolism by the liver and intestinal wall flora. Transmucosal drug dosage forms (e.g., tablet, suppository, ointment, pessary, membrane, and powder) are typically held in contact with the mucosal membrane and disintegrate and/or dissolve rapidly to allow immediate systemic absorption. Note that certain such routes may be used even where the patient is unable to ingest a treatment composition orally.

For delivery to the buccal or sublingual membranes, typically an oral formulation, such as a lozenge, tablet, or capsule, will be used. The method of manufacture of these formulations is known in the art, including, but not limited to, the addition of the pharmacological agent to a pre-manufactured tablet; cold compression of an inert filler, a binder, and either a pharmacological agent or a substance containing the agent (as described in U.S. Pat. No. 4,806,356); and encapsulation. Another oral formulation is one that can be applied with an adhesive, such as the cellulose derivative hydroxypropyl cellulose, to the oral mucosa, for example as described in U.S. Pat. No. 4,940,587. This buccal adhesive formulation, when applied to the buccal mucosa, allows for controlled release of the pharmacological agent into the mouth and through the buccal mucosa.

Parenteral administration is generally characterized by injection, either subcutaneously, intramuscularly, or intravenously. Thus, this invention provides compositions for intravenous administration that comprise a solution of a compound of the invention dissolved or suspended in an acceptable carrier. Injectables can be prepared in conventional forms, either as liquid solutions or suspensions, solid forms suitable for solution or suspension in liquid prior to injection, or as emulsions. Suitable excipients are, for example, water, buffered water, saline, dextrose, glycerol, ethanol, or the like. These compositions will be sterilized by conventional, well known sterilization techniques, such as sterile filtration. The resulting solutions can be packaged for use as is or lyophilized, the lyophilized preparation being combined with a sterile solution prior to administration. In addition, if desired, the pharmaceutical compositions to be administered may also contain minor amounts of non-toxic auxiliary substances, such as wetting or emulsifying agents, pH buffering agents and the like, such as for example, sodium acetate, sorbitan monolaurate, triethanolamine oleate, etc.

Another method of parenteral administration employs the implantation of a slow-release or sustained-release system, such that a constant level of dosage is maintained.

Liquid pharmaceutically administrable compositions can, for example, be prepared by dissolving, dispersing, etc., an active compound as defined above and optional pharmaceutical adjuvants in an excipient, such as, for example, water, saline, aqueous dextrose, glycerol, ethanol, olive oil, and other lipophilic solvents, and the like, to form a solution or suspension. If desired, the pharmaceutical composition to be administered may also contain minor amounts of nontoxic auxiliary substances, such as wetting or emulsifying agents, pH buffering agents, and the like, for example, sodium acetate, sorbitan monolaurate, triethanolamine sodium acetate, triethanolamine oleate, etc. Actual methods of preparing such dosage forms are known and will be apparent to those skilled in this art. The composition or formulation to be administered will contain an effective amount of a S100 protein inhibitor of the invention.

For solid compositions, conventional nontoxic solid carriers can be used and include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 0.1-95% of active ingredient, preferably about 20%. It will appear to those skilled in the art, or to pediatricians or doctors, that the dosage or S100 protein inhibitors will be depending on the state and nature of the leukemia to be treated and/or reduced, or of the combination with another chemotherapeutic compound.

The compositions containing the compounds of the invention can be administered for prophylactic and/or therapeutic treatments. In therapeutic applications, compositions are administered to a patient already suffering, for example, from CML, as described above, in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

In addition to body administration, the compounds and compositions of the invention may be applied, for example but not limited to, ex vivo to achieve therapeutic effects. In such an application, cells to be treated, e.g., blood or bone marrow cells, are removed from a patient and treated with a pharmaceutically effective amount of a compound of the invention. The cells are returned to the patient following treatment. Such a procedure can allow for exposure of cells to concentrations of therapeutic agent for longer periods or at higher concentrations than otherwise available.

Once improvement of the patient's conditions has occurred, a maintenance dose is administered if necessary. Subsequently, the dosage or the frequency of administration, or both, can be reduced, as a function of the systems, to a level at which the improved condition is retained. When the symptoms have been alleviated to the desired level, treatment can cease. Patients can, however, require additional treatment upon any recurrence of the disease symptoms.

In prophylactic applications (e.g. chemoprevention), compositions containing the compounds of the invention are administered to a patient susceptible to or otherwise at risk of developing a leukemia or CLL. Such an amount is defined to be a “prophylactically effective amount or dose.” In this use, the precise amounts again depend on the patient's state of health and weight.

The present invention will be more readily understood by referring to the following examples which are given to illustrate the invention rather than to limit its scope.

### EXAMPLE I

### Leukocyte Accumulation Induced by Murine S100A8 in the Air Pouch Model

Material and Methods

Cells Preparation

Peripheral blood and bone marrow cells (BM) from AML, CML patients and healthy donors (graciously given by Dr Robert Delage, Hôpital de l'Enfant-Jésus, Québec) were centrifuged to collect sera, then peripheral blood mononuclear cells (PBMC) were isolated using erythrocyte sedimentation by dextran 2% followed by Ficoll-paque gradient centrifugation. In AML and CML patients, the PBMC fraction was enriched in immature progenitors and precursor cells.

Recombinant S100 Proteins

Human S100 protein cDNA were cloned into the pET28 expression vector. Recombinant protein expression was induced with 1 mM IPTG in E. coli HMS174. The bacteria were then lysed by sonication and recombinant His-tag S100 proteins were purified using a nickel column. S100 proteins bound to the column were freed by incubation with biotinylated thrombin and were eluted with PBS containing 0.5 M NaCl. Streptavidin-agarose was added to remove the contaminating thrombin. Finally the protein solutions were passed through a polymyxin B-agarose column to remove endotoxins.

ELISA

Ninety-six-well plates were coated overnight at 4° C. with 100 μL of purified rabbit IgG against S100A12 or mouse mAb against S100A8/A9 (generous gift of Dr Nancy Hogg, Leukocyte Adhesion Laboratory, London Research Institute, London, U.K.). The plates were blocked with PBS, 1% Tween 20™, 2% bovine serum albumin (BSA) for 45 min at R.T. The sera and standards (100 μL) were added and incubated for 45 minutes at R.T. After three washes, the plates were incubated with 100 μL of rat anti-S100A12 polyclonal or rabbit anti-S100A8/A9 antisera for 45 minutes at R.T. Following incubation, the plates were washed three times and incubated with peroxidase-conjugated goat anti-rat or anti-rabbit for 45 minutes. After three washes, the presence of IgG was detected by addition of 3,3′,5,5′tetramethylbenzidine and the optical density was read at 450 nm. The detection limit was 10 ng/ml for S100A12 and 0.4 ng/ml for S100A8/A9.

Methyl Thiazole Tetrazolium (MTT) Growth Assay

PBMC or BM cells were cultured in 100 μL RPMI 1640 containing 10% foetal bovine serum in presence or absence of increasing concentrations of recombinant S100 proteins or different dilutions ( 1/80 to 1/10) of autologous bone marrow serum in presence or absence of purified mAb against S100A8 or S100A9. After 72 h, 10 μL of MTT 12 mM were added and samples were incubated 4 hours at 37° C. One hundred μL of SDS 0.01%-HCL 0.01N were added, the plates were incubated for 4 to 18 hours at 37° C. and the optical density was read at 600 nm.

Colony-Forming Unit (CFU)

PBMC and BM (5×10⁴ cells) were incubated at 37° C. in methylcellulose-based media containing 1% methylcellulose in Iscove MDM, 30% foetal bovine serum, 1% BSA, 10⁻⁴ M 2-mercaptoethanol and 2 mM of L-glutamine supplemented (+CSF) or not (−CSF) by 50 ng/mL of rhSCF, 10 ng/mL of rhIL-3, and 10 ng/mL of rhGM-CSF to support optimal growth of CFU-Granulocyte Macrophage (CFU-GM) CFU-Granulocyte (CFU-G) and CFU-Macrophage (CFU-M). Different concentrations of S100A12, S100A8, and/or S100A9 were also added to the cells. After 15 days, colony-forming units were counted.

Air pouches were raised in 10 to 12 week-old CD-1 mice. One ml of murine S100A8 (10 μg/ml, 5 nM) or phosphate buffered saline (PBS) were injected into the air pouches and the migrating leukocytes were harvested by washing with PBS-EDTA 5 mM at various times. Leukocytes in exudates were counted, centrifuged on microscope slides and stained with Wright-Giemsa. Data represent the mean±SEM of at least 7 mice (FIG. 1).

### EXAMPLE II

### Effect of Increasing Concentrations of Murine S100A812 on Circulating Neutrophils in Blood

Increasing doses of murine S100A12 or PBS were injected. i.v. into the tail vein of mice and blood was harvested by cardiac puncture 3 h later. Total leukocytes were counted using a haemocytometer following acetic blue staining. Cytospin preparations of leukocytes were analyzed after Wright-Giemsa staining. Data represent the mean±SEM of at least 6 mice for each group (FIG. 2). *p<0.05, **p<0.01, Dunnett multiple comparison test. It is observed from the present data that S100 protein S100A12 has a significant stimulatory activity on the proliferation of cells in the blood.

### EXAMPLE III

### Effect of S100A8 on Bone Marrow Cell Proliferation

Bone marrow cells were collected by flushing PBS through incisions made in the femurs of mice, followed by disaggregation. After removing contaminating erythrocytes by hypoosmotic lysis, bone marrow cells were cultured in semi-solid state in Methocult methylcellulose-based media (Stemcell technologies) for 14 days in presence of increasing concentrations of S100A8. At the end of the incubation period, the colonies were numerated. Similar results were obtained with S100A9, S100A8/A9, and S100A12. Results are from one experiment representative of 2 others (FIG. 3). All monomers and heterodimers have shown a growth stimulatory activity on bone marrow cells.

### EXAMPLE IV

### Effect of S100A8, S100A9, S100A8/A9, and S100A12 on CML Bone Marrow Cell Proliferation

Bone marrow cells were obtained by aspirates from a CML patient. (A) The cells were cultured for 14 days in Iscove medium supplemented with 15% FCS and 2% methylcellulose, in presence of increasing concentrations of S100A12. (B) The cells were culture for 14 days in presence of 10 pg/ml of S100A8, S100A9, S100A8/A9, or S100A12 in a methylcellulose medium supplemented or not with 50 ng/ml of Stem cell factor, 10 ng/ml of GM-CSF, and 10 ng/ml of IL-3. The results are the number of colonies per 30 mm² petri dishes (FIG. 4). As observed in Example III, different S100 proteins have stimulatory activity on bone marrow cells.

### EXAMPLE V

### Inhibition by Antibodies Against S100A8 and S100A9 of Leukemic Cell Proliferation Induced by the Serum from a Leukemic Patient

Peripheral blood mononuclear cells were obtained from a patient suffering from AML and cultured with a 1:10 dilution of his own serum in presence or absence of polyclonal antibodies against S100A8 and S100A9. The results given in FIG. 5 show that the inhibition of two members of the S100 family clearly allows to inhibit the proliferation of leukemia cells.

All patents, patent applications, articles and publications mentioned herein, both supra and infra, are hereby incorporated herein by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1-26. (canceled)

27. A method for reducing or inhibiting at least one of differentiation and development of progenitor blood cells into leukemia cells or the growth of leukemia cells in a human or an animal in need thereof, said method comprising the step of administering to the human or animal a composition comprising a pharmaceutically acceptable carrier and an effective amount of at least one inhibitor of a protein selected from the group consisting of a S100A8 protein and a S100A9 protein, resulting in the reduction or inhibition in at least one of the differentiation and development of said progenitor blood cells into leukemia cells or growth of leukemia cells in the human or animal.

28. The method of claim 27, wherein said progenitor blood cells are bone marrow stem cells.

29. The method of claim 27, wherein said progenitor blood cells differentiate or develop into leukocytes, neutrophils, eosinophils, basophils, lymphocytes, macrophages, or monocytes.

30. The method of claim 27, wherein said inhibitor is selected from the group consisting of an antibody, an antibody binding fragment thereof, a peptide, an anti-mRNA, an RNAi, an inhibitor of transcription of the gene encoding said protein, an inhibitor of translation of the mRNA encoding said protein, a binding inhibitor binding said protein, and a binding inhibitor binding the natural binding site of said protein.

31. The method of claim 27, wherein said S100A8 protein and said S100A9 protein are in homodimeric or hetero dimeric form.
